(19) United States
(12) Patent Application Publication
(10) Pub. No.: US 2005/0118133 A1
(43) Pub. Date: Jun. 2, 2005

(54) Active agent mixture containing a plant extract of convolvulaceae

(76) Inventor: Shaho Ismail Barzanji, Andazyaran (IQ)

Correspondence Address: THOMTE, MAZOUR & NIEBERGALL, L.L.C., 2120 S. 72ND STREET, SUITE 1111, OMAHA, NE, 68124, US

(21) Appl. No.: 10/499,797

(22) PCT Filed: Dec. 19, 2002
(86) PCT No.: PCT/DE02/04670

(30) Foreign Application Priority Data
Dec. 21, 2001 (DE) 101 63 190.1

(51) Int. Cl. A61K 7/06; A61K 35/78

(52) U.S. Cl. 424/74; 424/725

(57) ABSTRACT

The invention relates to an active agent mixture, obtained from plants of the Convolvulaceae family. According to the invention, the following are produced from the plants: (i) an aqueous decoction by boiling in water, or (ii) an alcoholic extract by steeping in alcohol, or (iii) a paste by processing the finely ground plant with more or less viscous liquids which are compatible with the skin.

### BACKGROUND OF THE INVENTION

1. Field of the Invention

The present invention relates to an active agent mixture for producing an agent for the therapeutic and/or cosmetic treatment of hair problems and/or other diseases and disorders of the skin.

2. Description of the Prior Art

Problems with the hair, whether it be hair loss, grey hair or lank thin hair, severely affect many of those concerned and thus considerably restrict their quality of life.

Skin diseases associated with itching, pigment disorders or other symptoms have a much more negative influence on the quality of the life of the persons concerned.

The prior art includes a number of agents provided to combat a few of said phenomena.

Various hair restorers are known, some of which are plant-based and gentle and environmentally friendly, whose effectiveness is however at most variable and generally not very satisfactory. Many act only specifically when there are specific causes for the hair loss, only a few have a wider-ranging effect when there are several causes. More or less severe side effects are common to most of these agents.

An agent which restores the original colour back to grey hair is know for grey hair. However, this agent does not have the effect that the hair continues to grow in its original colour. It also does not prevent other hair going grey.

There are numerous, specifically effective medicaments for skin diseases. Fungal infections, pigment disorders, pruritus and psoriasis can be treated more or less successfully with these agents, wherein more or less severe side effects must be accepted in each case. So far, there is still no agent which has a palliative or healing effect on all of said diseases.

### SUMMARY OF THE INVENTION

The object of the invention is to provide an agent which has a positive and wide-ranging effect as far as complete healing on the aforesaid hair and skin problems or diseases and in addition is free from or approximately free from side effects.

A solution of this object consists in providing an active agent mixture of the type specified initially, which is characterized in that the active agent mixture is obtained from plants of the Convolvulaceae family, wherein the following are produced from the plants: either (i) an aqueous decoction by boiling in water, or (ii) an alcoholic extract by steeping in alcohol, or (iii) a paste by processing the finely ground plant with more or less viscous liquids which are compatible with the skin.

As part of the studies which have resulted in the present invention, it was surprisingly found that this plant active agent mixture, hereinafter called Shaho preparation, stimulates and invigorates the skin to such an extent that the following effects are demonstrably achieved:
Stopping hair loss
Stimulating new hair growth
Vitalising existing hair roots
Stabilising and revitalizing hair pigment production
Normalising skin pigment production
Vitalising hair which is dry, thin or degenerated in other ways
Improving the symptoms of psoriasis as far as curing them.

The Shaho preparation acts on numerous diseases and disorders of the skin. is simple and environmentally friendly to produce, is well tolerated and has almost no side effects.

Combined with other simple plant active agents, the effect on particular diseases can be specifically intensified (as is described subsequently). The good tolerability allows preventative usage as well as usage for cosmetic purposes.

The active agent mixture of the Shaho preparation is preferably obtained from plants of the genus Convolvulus euphraticus, especially of the species Convolvulvus euphraticus Bornm.

The subject matter of the invention is thus an active agent mixture in itself which is characterised in that it is obtained from a plant of the species Convolvulus euphraticus Bornm., wherein the following are produced from the plant: either (i) an aqueous decoction by boiling in water, or (ii) an alcoholic extract by steeping in alcohol, or (iii) a paste by processing the finely ground plant with more or less viscous liquids which are compatible with the skin, wherein the decoction or the extract or the paste contains the active agent mixture.

In practical application it has been shown that the active agent mixture according to the invention is especially suitable for producing an agent for hair loss (a hair restorer), this agent showing a very good effectiveness.

The active agent mixture according to the invention can also be used specifically to produce an agent for vitalising and strengthening existing hair roots and thus for obtaining healthier, stronger and more beautiful hair.

Furthermore, the active agent mixture according to the invention is suitable for producing an agent which stimulates skin pigment production and thus stops the increase in grey hair and makes grey hair continue to grow in its original colour.

The active agent mixture according to the invention is also suitable for producing an agent which normalises skin pigment production and thus restores the original colour to areas of the skin which are either too light or too dark.

A further use of the active agent mixture according to the invention consists in producing an agent which is suitable for vitalising hair which is dry, thin or degenerated in other ways.

The active agent mixture according to the invention is not least especially provided for producing an agent for healing psoriasis, an agent for healing infectious diseases of the skin, an agent for ameliorating pruritus and an agent for ameliorating neurodermatitis.

The following advantages are especially associated with the active agent mixture according to the invention:
Hair loss was stopped in almost all probands (>90%) regardless of the cause. Both hereditary hair loss in men (androgenic alopecia) and other forms of hair loss in men and women could be stopped (including alopecia areate, so-called circular hair loss).
The best result was complete stoppage of hair loss after one week, the worst result was a reduction in hair loss by one third after three weeks.
In cases of hair loss caused by androgenic alopecia, success was usually seen within 10 days. Hair loss caused by alopecia areate was stopped within 4 to 8 weeks in all the probands affected.
New hair growth could be observed in over 90% of probands. Density, growth rate and required duration of treatment depended on how long the treated areas had already been bald.
Even on areas which had been bald for years, in almost all cases a surface-covering thin down was formed after three months, in addition to individual hairs 4 to 7 em long. In one individual case, after intermediate renewed hair loss, surface-covering thick growth of new strong healthy hair occurred six months after terminating the treatment.
In less severe cases, the success was correspondingly greater and could also be achieved more rapidly.
In all cases, after treatment for one to three weeks dull and lank thin hair had continued to grow more healthily, more strongly and more beautifully. This effect was maintained in the long term after the end of treatment.
Hair which had only turned grey over the last three months or less grew back again with the old colour after treatment for approximately ten days.
Greying of further hair was stopped or slowed down. On average approximately 90% of the hair retained its original colour permanently.
Skin pigment disorders, both light and dark spots, which had only occurred over the last few months could be healed after treatment for four to six months.
In all the probands the treatment showed a generally invigorating effect on the skin.
Thin areas of skin, skin with disorders of the sweat and sebaceous glands, and skin with degenerated connective and fatty tissue improved its condition with the duration of treatment. After treatment for 2 to 4 months at the latest these areas of skin had regained the capability of producing sweat and sebum in sufficient quantity.
In most probands the connective tissue had regained its old elasticity and this was after about 3. weeks.
The treatment of psoriasis was successful in all those affected. After 8 weeks at the latest the symptoms had disappeared.
Fungal infections of the skin were cured in all probands treated after about 3 to 4 weeks.
A positive effect on other infectious diseases of the skin is highly probable.
Pruritus as a result of allergies or, for example, as a result of extremely greasy scalp had disappeared after about 4 weeks in those persons who were treated.

### DESCRIPTION OF THE PREFERRED EMBODIMENT

The invention is explained in detail subsequently with reference to exemplary embodiments.

### EXAMPLE 1

### Production of an Alcohol-Free Shaho Preparation

All the parts of the plant above the ground are harvested from wild or cultivated plants of the family Convolvulaceae, preferably of the genus Convolvulus euphraticus and especially preferably of the species Convolvulus euphraticus Bornm. The preferred harvesting time is July to September.

The parts of the plant are used in freshly harvested or dried form. They should be washed thoroughly before processing.

The parts of the plant are boiled for 6 hours in boiling water, with 2.5 parts by 1 volume of water being added to 1 part by volume of loosely layered plant material.

During the boiling process the parts of the plant do not disintegrate but absorb a large part of the liquid, and the residual liquid is coloured green.

The liquid was separated from the solid plant material in a press at a pressure of 5 to 10 bar.

The liquid, i.e., the decoction, was further processed, namely thickened until it had approximately reached the viscosity of toothpaste (or slightly less liquid).

The thickening can be accomplished by boiling down or by evaporating at room temperature, in sunlight for example.

In proband tests it has been shown that decoction thickened by evaporation in sunlight achieves a higher effectiveness than decoction which has been boiled down.

### EXAMPLE 2

### Production of a Shaho Preparation Using Alcohol Extraction

Parts of plants are harvested and washed or cleaned as described in Example 1.

The plant parts are then cut into small pieces, steeped in 70 to 95 vol. % alcohol % alcohol in water) in the volume ratio 1:1 (loosely layered plant material to alcohol) and kept for 15-20 days (or longer) in an airtight closed container.

The plants do not disintegrate in this case but absorb a large part of the liquid.

The residual liquid is coloured brown.

The liquid is then separated from the solid plant material in a press at a pressure of approximately 5 bar. The liquid, i.e., the decoction, is then further processed.

The decoction is either used directly as liquid Shaho preparation (tincture) (see Example 4, “Shaho-F”) or is thickened by drying in the sun and/or air to such an extent that it has approximately reached the viscosity of toothpaste or is slightly less liquid.

### EXAMPLE 3

### Production of a Shaho Preparation by Grinding

Parts of plants are harvested and washed or cleaned as described in Example 1. The plant˜parts are then pulverised mechanically and ground into a paste.

### EXAMPLE 4

### Production of Shaho Cream (Shaho Ointment) and Shaho Liquid Preparations for Application in Proband Tests

Shaho-A (Cream/Ointment)

100 parts by weight of the thickened decoction manufactured according to Example 1 or Example 2 is mixed with 100 parts by weight of a natural fat (e.g. vegetable oils or animal fats) and 35 parts of weight of a cream or ointment base material.

The fat is used for the purpose of increasing the compatibility of the cream with the skin and for further promoting the drawing of the active agents into the skin.

Shaho-B (Cream/Ointment)

100 parts by weight of Shaho cream A is mixed with 25 to 50 parts by weight of garlic juice.

Shaho cream B is particularly effective for the treatment of circular hair loss.

Shaho-C (Cream/Ointment)

100 parts by weight of Shaho cream A is mixed with 25 parts by weight of garlic juice and 30 parts by weight of apple vinegar.

Shaho cream C is particularly effective for the treatment of psoriasis.

Shaho-E (Liquid Preparation)

The thickened decoction manufactured according to Example 1 or example 2 is mixed with distilled water in the mixing ratio of 1 part by weight of water to 4 to 10 parts by weight of decoction to form a highly fluid preparation. To this is added 2% (of the total weight of water and decoction) of vaseline or cream base fat and this is agitated thoroughly with the decoction-water mixture. Before use, this liquid preparation should or must be agitated again thoroughly.

Highly fluid preparations have a lower concentration of active agent but can be applied more easily between hairs still present on the scalp. For this reason, highly fluid Shaho-E preparations are especially provided for use in patients with little hair loss and a large amount of hair still present or in patients with thin hair and the disorders dandruff or itchy scalp.

More viscous Shaho-E preparations on the other hand are especially suitable for patients with little or no hair since in these cases a large amount of active agent is desired and application to the scalp presents no problem.

Shaho-F (Liquid Preparation)

The decoction manufactured according to example 2 and not yet thickened is diluted with distilled water in the ratio 4:1 (4 parts decoction+1 part water) and used in this form as liquid preparation Shaho-F. This Shaho-F is easy to use (because it is relatively highly fluid), rapidly reaches the hair roots and when applied daily (8-hour working duration) already shows the first positive effects within 7 days.

In principle, the liquid preparation Shaho-F has the same advantages as the liquid preparation Shaho-E.

Both preparations are especially provided for use in those patients who have only slight hair loss and are not prepared to shave off their hair for treatment.

The fact that Shaho-E and Shaho-F are easy to use also allows preventative application.

### EXAMPLE 5

### Proband Test Using Shaho-A, Shaho-B and Shaho-C Cream Preparations

In the proband test the Shaho-A, Shaho-B and Shaho-C cream preparations manufactured in accordance with Example 4 were successfully used for hair loss, for vitalising existing hair roots, for ameliorating pruritus caused by allergies and other factors and for revitalising hair pigment production.

The probands (test subjects) were divided into Grades 1 (slight) to 7 (very severe) according to the severity of the hair loss.

Most of the probands had had their hair shaved off for the duration of the treatment. Only one woman and four men with hair loss grade 1, grade 2 and grade 3 did not have their hair shaved off.

Before applying the preparation, the scalp was massaged for 5 to 10 minutes, then the head was washed and completely dried, in some cases blow-dried. The preparation was then applied directly onto the scalp and massaged in (existing hair was combed aside). Depending on the area to be treated, 1 to 5 cm3 of the preparation was applied, the 5 cm3 quantity being applied when treating the complete head. During the working time, heat treatment was carried out, e.g. by applying a warm head covering or by visiting a sauna.

The working time per treatment was generally between 1 and 8 hours. One hour was sufficient when a sauna was visited as heat treatment. Eight hours (e.g. one night) was completely sufficient in most cases. Seventy two hours was the maximum working time and was used for women with strong long hair.

(Experience shows that the active agents are absorbed the slowest in this group of people).

After the end of the working time (see below), the preparation was washed out.

For working times up to 8 hours, treatment was carried out each day and for a working time of 72 hours the treatment was carried out once a week.

The results of these tests are presented in Tables 1-7. The tables are each compiled for one degree of severity of the hair loss.

In these (and the other tables):

“Person No.” means the file number of the test documentation;
“A”, “B” or “C” in the “type” column denote the preparation Shaho-A or Shaho-B or Shaho-C;
“+” in the “nutrition” column means that in addition to the treatment with this Shaho preparation, a special nutrition program has been followed, “−” in this column means that no such program has been followed;
“+” in the “sport” column means that in addition to the treatment with this Shaho preparation, a special sport or movement program has been followed, “−” in this column means that no such program has been followed;
“Shave” stands for shaving off the hair for the duration of the treatment;
In the “shave” or “shaving” column “+” means that the scalp has been completely shaved smooth for the duration of the treatment and “−” means that no shaving has taken place;
“Quantity in cm” means the total quantity of the preparation given to the probands for the duration of treatment as well as the number and size of the individual portions; for example “45+45” in this column stands for “2 portions of 45 cm3”.
The duration of treatment is partly subdivided into individual blocks of several successive days. These blocks can directly follow one another or be separated by days to several months. No information on such intervals is given in the table. For example, “30+30” in the column concerned “duration of treatment in days” stands for 2 blocks of 30 days with an unknown interval between treatments.
“+” in the “effect” column means “effect demonstrably present”, “−” in this column-means “no effect identifiable”.
   In general, it holds that the best effect is achieved if existing hair is completely shaved off and the preparation can act under additional heat. A warm cap, warm sun or a visit to the sauna intensify the effect appreciably. The working time in this case is of the order of magnitude of hours up to three days. The longer the preparation can act on the skin, the better the effect. Massaging the preparation in improves the circulation of the skin and considerably increases the effect.

Experience shows that: men require a shorter working time than women, strong hair (e.g. during treatment for grey hair) requires a longer working time than the treatment of bald spots.

The frequency of treatment and the total duration of treatment can be matched individually to the respective patients.

### EXAMPLE 6

### Proband Tests Using Shaho-E and Shaho-F Liquid Preparations

In the proband test the Shaho E liquid preparation manufactured in accordance with Example 4 was successfully used for hair loss, for vitalising existing hair roots, for ameliorating pruritus caused by allergies and other factors and for revitalising hair pigment production.

The probands (one woman and four men with hair loss Grade 1, Grade 2 and Grade 3) did not have their hair shaved off. The scalp was massaged once a day, Shaho-E was applied and allowed to act for approximately 8 hours.

The result of this test is presented in Table 8.

The liquid preparation Shaho F maDufactured according to Example 4 was used in direct comparison to this in the proband test. The test was carried out under the same conditions as described for the liquid preparation Shaho E.

The result of this test is presented in Table 9.

### EXAMPLE 7

### Proband Test on the Application of Shabo Cream Preparations Especially for Women with Hair Loss

Especially for women (but naturally also for men) who do not wish to shave off their existing hair, the following procedure is proposed to combat hair problems:

In each treatment, 15-20 cm3 of a Shaho cream preparation (A, B or C) manufactured according to Example 4 is applied to the scalp, massaged in and allowed to work for 24-72 hours. During the working time heat treatment should take place, for example, by wearing a warm head covering.

With a working time of 72 hours the treatment is repeated twice more at intervals of 10 days (i.e., three times in one month).

In the proband test a total of 13 women of different ages and with different causes of hair loss were treated accordingly. The precise treatment plan and the results are presented in Table 10. It can be seen from this table that the treatment was very successful in all the probands: in most cases the hair loss was completely stopped and intensified hair regrowth was seen.

### EXAMPLE 8

### Proband Test for Treatment of Psoriasis

Two probands (No. 32 and No. 44) who were suffering from psoriasis on the scalp and additionally from androgenous hair loss were treated with the Shaho cream preparation Shaho-C manufactured in accordance with Example 4, over a period of several days. The precise treatment plan and the results are presented in Tables I-and 5. In these cases of disease a significant improvement in the condition was also achieved by treatment with the Shaho preparation: a significant improvement in the psoriasis symptoms was achieved both for person No. 32 with hair loss grade 5 (see Table 5) and for person No. 44 with hair loss grade 1 (see Table 1).

### EXAMPLE 9

### Proband Test for Treatment of Circular Hair Loss (Alopecia)

Twelve people affected by alopecia with different degrees of severity were treated in the proband test. The probands were treated with the Shaho cream preparation Shaho-B, manufactured in accordance with Example 4, over a period of 7 to 30 days. The precise treatment plan and the results are presented in Table 11. It can be seen from this Table 11 that the treatment was successful in all probands:

The symptoms of Alopecia greata could be identified in 8 probands. Of these two probands each exhibited a circle having a diameter of 2-3 cm and the time of infection in both these cases was less than one month. After the treatment hair loss and spreading of the circle had stopped and 100% new hair had re-grown. One proband suffered for more than six months with five circles each having a diameter of around 1 cm. As a result of the treatment hair loss and spreading of the circle stopped 100% and the first soft hair grew back. Two years later this proband was sprayed with hydrocortisone in the area of the original alopecia circle and 100% hair regrowth was thereby achieved. In another proband who was suffering from several large bald alopecia circles and had been affected by the disease for many years, spreading of the circles stopped 100% and new hair grew back. In another proband who had been suffering from the disease for two years, the alopecia circle at the beginning of treatment had a diameter of approximately 15 centimetres. As a result of the treatment with the Shaho preparation, further spreading of the circle was stopped 100%, the pores opened completely and three years after the treatment, the affected area had become significantly smaller, its diameter was only around 8 cm and the first soft hair had already frown back in the edge zone. One of the probands was affected by small circles of wet wounds, itching and fungal infection on the scalp. After treatment for one month, the hair loss was completely stopped, the itching had stopped, the wounds had disappeared and the hair grew back 100% in these circles. One proband gave no information on the effects of the treatment, and another proband stated verbally that the treatment had helped them.

The symptoms of Alopecia areata totalis were identifiable in one proband. This person was suffering from complete hair loss, their scalp was relatively immobile, the skin pores were completely closed, wounds with pruritus were present on the affected areas and the skin was very greasy. Treatment with Shaho preparation B showed the following effects: the pores opened completely, the normal thickness of the scalp was restored and the mobility of the scalp increased, the wounds healed completely, the pruritus disappeared, the first soft hair grew back and significantly fewer fatty deposits were present.

In the drawings and in the specification, there have been set forth preferred embodiments of the invention and although specific items are employed, these are used in a generic and descriptive sense only and not for purposes of limitation.

Changes in the form and proportion of parts, as well as a substitution of equivalents, are contemplated as circumstances may suggest or render expedient without departing from the spirit or scope of the invention as further defined in the following claims.

Thus it can be seen that the invention accomplishes at least all of its stated objectives.

| | | | | | Maner of Use | | | | | | | |
|---|---|---|---|---|---|---|---|---|---|---|---|---|
| Persons | | Shaho Cream | | | | | Head | Hair loss stopping | | Other Benefites | | |
| | Brith | | Amount | Period of Use/ | | | shaving | | After how many | Hair | Vitalisierung of | Skin |
| No. | Day | Type | (CC) | Day | Sport | Food | with razer | Rate % | Days | appearance | existing hair roots | diseases |
| 20 | 1972 | A | 45 | 30 | + | + | − | 90 | 7 | + | + | + |
| 28 | — | A | 30 | 14 | − | − | − | 100 | 15 | + | + | + |
| 41 | — | A | 45 | 30 | − | − | + | 100 | 15 | + | + | + |
| 44 | 1972 | A | 45 | 30 | − | − | − | 90 | 14 | + | + | + |
| 50 | 1972 | A | 45 | 30 | − | − | − | 75 | 14 | + | + | + |
| 53 | 1968 | A | 45 | — | − | − | − | − | − | + | + | + |
| 54 | — | A | 45 | 30 | − | − | − | − | − | + | + | + |
| 57 | 1950 | A | 90 | 14 | − | − | − | 95 | − | + | + | + |
| 61 | — | A | 45 | — | − | − | − | 95 | − | + | + | + |
| 66 | — | A | 12 | — | − | − | − | − | − | + | + | + |
| 71 | — | A | 30 | — | − | − | − | − | − | + | + | + |
| 74 | — | A | 12 | — | − | − | − | − | − | + | + | + |
| 101 | 1978 | A | 45 | 30 | − | + | − | 95 | − | + | + | + |
| 67 | 1969 | A | 45 | 25 | − | − | − | 90 | 30 | − | − | − |
| 72 | 1975 | A | 12 | 10 | − | − | − | 100 | − | + | + | + |
| 89 | 1981 | A | 20 | 7 | − | − | + | 100 | 10 | + | + | + |
| 90 | 1978 | A | 45 | 30 | − | − | + | 95 | − | + | + | + |
| 91 | 1979 | A | 12 | 7 | − | − | − | 60 | 14 | + | + | + |
| 92 | 1970 | A | 12 | 7 | − | − | + | 100 | 15 | + | + | + |
| 93 | 1973 | A | 30 | 25 | − | − | + | 100 | 30 | + | + | + |
| 94 | 1973 | A | 12 | 10 | − | − | − | 95 | 30 | + | + | + |
| 95 | 1977 | A | 12 | 8 | − | − | + | 90 | − | + | + | + |

| | | | | | Maner of Use | | | | | | | |
|---|---|---|---|---|---|---|---|---|---|---|---|---|
| | | Shaho Cream | | | | | Head | Hair loss stopping | | Other Benefites | | |
| Persons | | | | Period | | | shaving | | After | Hair | Vitalisierung | |
| | Brith | | Amount | of | | | with | Rate | how many | appear- | of existing | Skin |
| No. | Day | Type | (CC) | Use/Day | Sport | Food | razer | % | Days | ance | hair roots | diseases |
| 2 | 1948 | A | 45 + 45 + 45 | 20 + 15 + 20 | − | − | + | 100 | 7 | + | + | + |
| 3 | 1964 | A | 90 + 45 | 60 + 30 + 6 | − | + | − | 100 | 30 | + | + | + |
| 14 | 1970 | A | 45 | 25 + 25 | + | + | − | 75 | 15 | + | + | + |
| 15 | 1975 | A(1/2) | 45 | 30 | + | + | − | 20 | 14 | + | + | + |
| 16 | 1970 | A | 45 | 30 | − | − | − | 100 | 14 | + | + | + |
| 25 | 1974 | A | 45 | 40 | + | + | + | 95 | − | + | + | + |
| 26 | 1962 | A | 45 | — | − | + | − | − | − | + | + | + |
| 31 | 1967 | A | 45 | 30 | + | + | + | 100 | − | + | + | + |
| 33 | 1965 | A | 45 | 23 | + | + | − | 90 | | + | + | + |
| 35 | — | A | 45 | 30 | + | − | − | 100 | | | | |
| 43 | 1978 | A | 45 | 31 | + | − | − | 100 | − | + | + | + |
| 46 | 1974 | A | 45 | — | − | − | − | − | − | + | + | + |
| 56 | — | A | 45 | — | − | − | − | − | − | + | + | + |
| 63 | 1980 | A | 20 | 10 | − | + | − | 100 | − | + | + | + |
| 68 | 1942 | A | 45 + 45 | — | − | − | − | − | − | + | + | + |
| 75 | — | A | 12 | — | − | − | − | | − | + | + | + |
| 103 | 1975 | A | 50 | 30 | − | − | − | | | + | + | + |
| 88 | 1965 | A | 20 | 12 | − | − | + | 100 | 15 | + | + | + |
| 82 | 1956 | A | 12 | 10 | − | − | + | 100 | 15 | + | + | + |
| 83 | 1966 | A | 14 | 14 | − | − | + | 90 | 60 | + | + | + |
| 85 | 1974 | A | 12 | 12 | − | − | + | 85 | 15 | + | + | + |
| 96 | 1972 | A | 20 | 12 | − | − | + | 80 | 15 | + | + | + |
| 97 | 1975 | A | 50 | 30 | − | − | + | 100 | 15 | + | + | + |

| | | | | | Maner of Use | | | | | | | |
|---|---|---|---|---|---|---|---|---|---|---|---|---|
| | | Shaho Cream | | | | | Head | Hair loss stopping | | Other Benefites | | |
| Persons | | | | Period | | | shaving | | After | Hair | Vitalisierung | |
| | Brith | | Amount | of | | | with | Rate | how many | appear- | of existing | Skin |
| No. | Day | Type | (CC) | Use/Day | Sport | Food | razer | % | Days | ance | hair roots | diseases |
| 9 | 1973 | A | 45 | 32 | + | + | − | 0 | − | + | − | − |
| 12 | — | A | 45 | — | − | − | − | + | − | + | + | + |
| 13 | 1973 | A | 45 + 45 + 45 | 45 + 8 + 3 + 2 | + | + | − | 100 | 30 | + | + | + |
| 17 | 1965 | A | 45 + 45 | 30 + 20 | + | + | − | 85 | 21 | + | + | + |
| 37 | — | A | 12 | 7 | − | − | − | 75 | 8 | + | + | + |
| 42 | 1960 | A | 45 | 30 | + | + | − | 100 | 1 | + | + | + |
| 47 | — | A | 30 | — | − | − | − | − | − | − | − | − |
| 48 | — | A | 45 | 30 | − | − | | 100 | + | + | + | + |
| 49 | — | A | 45 | — | − | − | − | − | − | − | − | − |
| 60 | — | C | 60 | — | − | − | | 100 | − | + | + | + |
| 70 | — | A | 12 | 5 | − | − | + | | | | | − |
| 78 | 1980 | A | 25 | 15 | − | − | + | 100 | 5 | + | + | + |
| 79 | 1963 | A | 45 | 7 | − | − | + | 95 | 15 | + | + | + |
| 86 | 1974 | A | 15 | 30 | − | − | + | 98 | 7 | + | + | + |
| 98 | 1972 | A | 25 | 14 | − | − | + | 95 | 14+ | + | + | + |

| | | | | | Maner of Use | | | | | | | |
|---|---|---|---|---|---|---|---|---|---|---|---|---|
| | | Shaho Cream | | | | | Head | Hair loss stopping | | Other Benefites | | |
| Persons | | | | Period | | | shaving | | After | Hair | Vitalisierung | |
| | Brith | | | of | | | with | Rate | how many | appear- | of existing | Skin |
| No. | Day | Type | Amount (CC) | Use/Day | Sport | Food | razer | % | Days | ance | hair roots | diseases |
| 18 | 1971 | A | 45 + 45 | 30 | + | + | − | 70 | 20 | + | + | + |
| 19 | 63 | A | 45 + 45 + 45 + 12 | 30 + 30 + 30 + 7 | + | + | + | 95 | 15 | + | + | + |
| 29 | 1971 | A | 45 + 45 + 45 + 45 | 30 + 30 + 30 + 30 | + | − | + | 90 | 17 | + | + | + |
| 39 | 1971 | A | 45 | 30 | − | − | − | 95 | 15 | + | + | + |
| 58 | 1951 | A | 45 | 7 | − | − | − | 60 | 60 | + | + | + |
| 62 | — | A | 12 | 2 + 2 | | | − | | | | | |
| 73 | | A | 45 | | − | − | | | | + | − | −− |
| 81 | 1965 | A | 12 | 7 | − | − | + | 80 | 10 | + | + | + |
| 87 | 1968 | A | 12 | 15 | − | − | + | | 30 | + | + | + |

| | | | | | Maner of Use | | | | | | | |
|---|---|---|---|---|---|---|---|---|---|---|---|---|
| | | Shaho Cream | | | | | Head | Hair loss stopping | | Other Benefites | | |
| Persons | | | | Period | | | shaving | | After | Hair | Vitalisierung | |
| | Brith | | | of | | | with | Rate | how many | appear- | of existing | Skin |
| No. | Day | Type | Amount (CC) | Use/Day | Sport | Food | razer | % | Days | ance | hair roots | diseases |
| 5 | 1968 | A | 45 + 45 + 45 + 45 | 20 + 25 + 40 + 25 | − | − | − | 100 | + | + | + | + |
| 7 | 57 | A | 45 | 30 | + | + | − | 100 | + | + | + | + |
| 11 | 73 | A | 45 + 45 + 45 | 20 + 2 + 30 + 3 | + | − | − | 90 | + | + | + | + |
| 22 | 1965 | A | 45 + 45 | 30 + 27 + 30 + 30 | + | + | − | 100 | | | − | |
| 27 | 1956 | A | | | | | | | + | + | + | + |
| 32 | 1973 | A | 45 + 45 | 30 + 30 | + | + | + | 100 | + | + | + | + |
| 38 | 1972 | A | 45 + 45 | 30 + 30 | + | −− | − | 90 | + | + | + | + |
| 40 | 1965 | A | 45 + 45 | 25 | + | + | − | 100 | | | | |
| 65 | | A | — | | | | | | | | | |
| 69 | | A | | 12 | − | | | | | | | |
| 73 | | A | 12 | — | | | | | | | | |
| 77 | | A | 45 | — | | | | | | | | |
| 99 | 1971 | A | 45 | 15 | − | − | + | 100 | 15 | + | + | + |

| | | | | | Maner of Use | | | | | | | |
|---|---|---|---|---|---|---|---|---|---|---|---|---|
| | | Shaho Cream | | | | | Head | Hair loss stopping | | Other Benefites | | |
| Persons | | | | Period | | | shaving | | After | Hair | Vitalisierung | |
| | Brith | | Amount | of | | | with | Rate | how many | appear- | of existing | Skin |
| No. | Day | Type | (CC) | Use/Day | Sport | Food | razer | % | Days | ance | hair roots | diseases |
| 30 | | A | 45 + 45 + 45 | | | | | 100 | | | | |
| 34 | | A | 45 | 30 | | | | − | | | | |
| 36 | 52 | A | 45 + 45 + 45 | 2 + 30 + 20 | | | | − | | | | |
| 51 | | D | 45 | 7 | | | | − | | + | − | |
| 80 | 1971 | A | 12 | 9 | − | − | + | 90 | + | + | + | + |

| | | | | | Maner of Use | | | | | | | |
|---|---|---|---|---|---|---|---|---|---|---|---|---|
| Persons | | Shaho Cream | | | | | Head | Hair loss stopping | | Other Benefites | | |
| | Brith | | Amount | Period of Use/ | | | shaving | | After how many | Hair | Vitalisierung of | Skin |
| No. | Day | Type | (CC) | Day | Sport | Food | with razer | Rate % | Days | appearance | existing hair roots | diseases |
| 1 | 1956 | A | 45 | 9 | − | − | − | 90 | 30 | | − | |
| 4 | 1954 | A | 45 + 45 | 60 | − | + | − | | | | | |
| 6 | 1959 | A + B | 45 + 45 | 30 + 30 | − | + | − | | | | | |
| 8 | 1956 | A | 45 | 15 | − | + | − | 30 | 7 | | | |
| 10 | 1946 | A | 45 + 45 | 60 + 20 | + | − | − | 100 | 7 | | | |
| 21 | 1962 | A | 45 + 45 | 30 | − | − | − | | | | | |
| 23 | 1947 | A | 45 + 45 | 30 + 30 | − | + | − | 25 | | | | |
| 24 | 1957 | A | 45 | 30 | − | − | − | 100 | 14 | | | |
| 52 | | D | 45 | — | − | − | − | | | − | | |
| 78 | 1936 | D | 45 | — | − | − | − | | | | | |
| 59 | 1954 | A | 10 | 5 | − | − | − | + | + | + | + | + |
| 84 | 1953 | A | 45 | 30 | − | − | − | + | − | + | + | + |

| | | | Vitalisierung of | | | | |
|---|---|---|---|---|---|---|---|
| | Hair loss | Hair loss | existing hair | Hair | Period of Use/ | | |
| No. | stopping | Grades | roots | appearance | Day | Other Benefites | Gender/Age |
| 1 | 90% | 2 | + | + | 15 | Jucken + | M 24 |
| | | | | | | Schuppen geheilt | |
| 2 | 85% | 1 | + | − | 7 | | M 35 |
| 3 | 100% | 3 | + | − | 20 | | M 38 |
| 4 | 95% | 2 | + | + | 20 | Jucken + Pickel | M 28 |
| | | | | | | geheilt | |

| | | | Vitalisierung of | | | | |
|---|---|---|---|---|---|---|---|
| | Hair loss | Hair loss | existing hair | Hair | Period of Use/ | | |
| No. | stopping | Grades | roots | appearance | Day | Other Benefites | Gender/Age |
| 1 | 100% | 2 | + | + | 15 | | M 28 |
| 2 | 90% | 2 | + | − | 7 | Jucken + | M 26 |
| | | | | | | schuppen geheilt | |
| 3 | 95% | 1 | + | − | 7 | | M 38 |
| 4 | 100% | — | + | + | 20 | Jucken + Pickel | M 28 |
| | | | | | | geheilt | |
| 5 | 100% | — | + | ++ | 15 | — | F 35 |
| 6 | 100% | — | + | ++ | 15 | | F 33 |

| | | | | Benefites | | |
|---|---|---|---|---|---|---|
| | | Shaho Cream | | Hair | | Vitalisierung |
| Persona | | | Amount | loss | Hair | of existing |
| No. | Age | Type | (CC) | stopping | appearance | hair roots |
| 1 | 50 | A | 20 | 100% | + | + |
| 2 | 45 | A | 20 | 95% | + | + |
| 3 | 35 | A | 20 | 85% | + | + |
| 4 | 27 | A | 20 | 100% | + | + |
| 5 | 17 | A | 20 | 100% | + | + |
| 6 | 25 | A | 20 | 100% | + | + |
| 7 | 35 | A | 20 | 100% | + | + |
| 8 | 32 | A | 20 | 95% | + | + |
| 9 | 38 | A | 20 | 100% | + | + |
| 10 | 18 | A | 20 | 100% | + | + |
| 11 | 19 | A | 20 | 100% | + | + |
| 12 | 75 | A | 20 | 100% | − | − |
| 13 | 40 | A | 20 | 85% | + | + |

| | | | | | | Merits (benefits) | | | |
|---|---|---|---|---|---|---|---|---|---|
| | Number | Diameter | Duration | Treatment | | Stop widen- | Skin | Re-grow | Other merits |
| S. No. | of spots | of spotted | of infection | Amount/cc | Usage time/ | ing spot | refreshment | of | (benefits) |
| 1 | 1 | 15 | 2 years | 50 | 20 | 100% | Complete | Few | |
| 2 | 1 | 2 | 1 month | 6 | 7 | 100% | Complete | 100% | |
| 3 | 4 | 2, 4, 7 | 4 years | 50 | 15 | 100% | Few | Very few | |
| 4 | All scalp | | 3 years | 50 | 25 | | Complete | Very few | Heat itching in scalp |
| 5 | All scalp, | | 4 years | 50 | 25 | | A lot | | |
| | eye brows, | | | | | | | | |
| | eye shield, | | | | | | | | |
| | beard and | | | | | | | | |
| | mustaches | | | | | | | | |
| 6 | Spots, wounds | 5 | ½ | 50 | 15 | 100% | Complete | 100% | Heal wounds, remove |
| | fungy | | | | | | | | itching and fungi |
| | | | | | | | | | spots in scalp |
| 7 | Upper part of | | 3 years | 125 | 60 | 100% | Complete | Few | After 1 year passed |
| | the scalp with | | | | | | | | over the treatment |
| | existence of | | | | | | | | he took local cortisone |
| | high ratio of | | | | | | | | ampoule in spots from |
| | fat and few | | | | | | | | specialist doctor, the |
| | number of hair | | | | | | | | hair grow in that |
| | spread on both | | | | | | | | spot 100%. |
| | sides of the | | | | | | | | |
| | head. | | | | | | | | |
| 8 | 5 | 1 | 2 years | 20 | 7 | 100% | * | Very few | |
| 9 | 1 | 3 | 1 month | 6 | 10 | 100 | * | 100% | |
| 10 | No answer | | | | | | | | |
| 11 | 3 | 3, 3, 5 | 3 years | 50 | No answer | | | | |
| 12 | Complete scalp | | 10 years | 75 | 30 | 100% | few | * | Flexibility came |
| | dryness with | | | | | | | | back to the scalp |
| | existence of | | | | | | | | |
| | few number of | | | | | | | | |
| | hair spread | | | | | | | | |
| | on both sides | | | | | | | | |
| | of the head. | | | | | | | | |

1. An active agent mixture for producing an agent for the therapeutic and/or cosmetic treatment of hair problems and/or other diseases and disorders of the skin, characterised in that the active agent mixture is obtained from plants of the Convolvulaceae family, wherein the following are produced from the plants: either (i) an aqueous decoctlon by boiling in water, or (ii) an alcoholic extract by steeping in alcohol, or (iii) a paste by processing the finely ground plant with more or less viscous liquids which are compatible with the skin.

2. The active agent mixture according to claim 1, characterised in that said active agent mixture is obtained from plants of the genus Convolvulus euphraticus.

3. The active agent mixture according to claim 1, characterised in that said active agent mixture is obtained from plants of the species Convolvulus euphraticus Bornm.

4. An active agent mixture characterised in that it is obtained from a plant of the species Convolvulus euphraticus Bornm. wherein the following are produced from the plant: either (i) an aqueous decoction by boiling in water, or (ii) an alcoholic extract by steeping in alcohol, or (iii) a paste by processing the finely ground plant with more or less viscous liquids which are compatible with the skin, wherein the decoction or the extract or the paste contain the active agent mixture.

5. The active agent mixture according to anyone of claims 14, characterised in that this is used to produce an agent for hair loss (a hair restorer).

6. The active agent mixture according to anyone of claims 1-4, characterised in that this is used to produce an agent which stimulates skin pigment production and thus stops the increase in grey hair and makes grey hair continue to grow in its original colour.

7. The active agent mixture according to anyone of claims 14, characterised in that this is used to produce an agent for healing psoriasis.

8. The active agent mixture according to anyone of claims 14, characterised in that this is used to produce an agent for healing infectious diseases of the skin.

9. The active agent mixture according to anyone of claims 1-4, characterised in that this is used to produce an agent for ameliorating pruritus.

10. The active agent mixture according to anyone of claims 1-4, characterised in that this is used to produce an agent for ameliorating neurodermatitis.
